(19) United States
(12) Patent Application Publication
(10) Pub. No.: US 2016/0287118 A1
(43) Pub. Date: Oct. 6, 2016

(54) COMPUTATIONAL TOOL FOR PRE-SURGICAL EVALUATION OF PATIENTS WITH MEDICALLY REFRACTORY EPILEPSY

(71) Applicants: The Johns Hopkins University, Baltimore, MD (US); THE CLEVELAND CLINIC FOUNDATION, Cleveland, OH (US)

(72) Inventors: Sridevi Sarma, McLean, VA (US); Bhaskar Chennuri, Baltimore, MD (US); John T. Gale, Chardon, OH (US); Jorge Alvaro Gonzalez-Martinez, Moreland Hills, OH (US)

(73) Assignees: The Johns Hopkins University, Baltimore, MD (US); THE CLEVELAND CLINIC FOUNDATION, CLEVELAND, OH (US)

(21) Appl. No.: 15/089,058

(22) Filed: Apr. 1, 2016

Related U.S. Application Data
(60) Provisional application No. 62/141,698, filed on Apr. 1, 2015.

(51) Int. Cl. A61B 5/04; A61B 5/00; A61B 5/0478

(52) U.S. Cl. CPC A61B 5/04012; A61B 5/0478; A61B 5/4094

(57) ABSTRACT

A method of identifying an epileptogenic zone of a brain includes receiving a plurality of electrical signals from a plurality of surgically implanted electrodes, calculating components of an adjacency matrix, calculating eigenvectors from the adjacency matrix, and selecting an eigenvector having a largest eigenvalue. The method includes assigning an integer rank to each component of the eigenvector, sliding the time window by a time increment and repeating the immediately preceding steps a plurality of times. The method includes normalizing each rank signal, extracting a multidimensional feature vector from each normalized signal, projecting each multidimensional feature vector onto a reduced dimensionality space, and receiving a plurality of training data points represented in the reduced dimensionality space. The method includes calculating grid weights for each feature vector, and assigning a numerical value to each electrode as an indication of whether the electrode is in an epileptogenic zone of the brain.

### CROSS-REFERENCE OF RELATED APPLICATION

This application claims priority to U.S. Provisional Application No. 62/141,698 filed Apr. 1, 2015, the entire content of which is hereby incorporated by reference.

### BACKGROUND

1. Field of Invention

The field of the currently claimed embodiments of this invention relates to methods and systems for evaluating epileptic zones of a subject's brain.

2. Discussion of Related Art

Epilepsy is one of the most common brain disorders, characterized by chronically recurrent seizures resulting from excessive electrical discharges from groups of neurons (1). Epilepsy affects about 50 million people worldwide and over 30% of all individuals with epilepsy have intractable seizures, which cannot completely be controlled by medical therapy (2-4). That is, seizures continue to occur despite treatment with a maximally tolerated dose of at least two anti-epilepsy drugs (AEDs). The direct cost of assessing and treating patients with medically refractory epilepsy (MRE) ranges from $3-4 billion annually 16 ($billion in direct and indirect costs) in the US (based on a 1996 publication) (5). 80% of these costs are accounted by patients whose seizures are not adequately controlled by AEDs (6). The burden of MRE, however, is much greater than heavy financial costs. MRE is a debilitating illness where individuals lose their independence, causing profound behavioral, psychological, social, financial, and legal issues (7-11). Recurrent seizures impair socialization and psychological development during formative years and may lead to an inability to obtain an education, gainful employment, or driving privileges. The development of a learned helplessness and low self-esteem can worsen as long as epilepsy is intractable. Cognitive performance may be impaired by MRE as well as by side effects of AED therapy (7-11).

Despite the heavy sequelae from MRE, there is a potentially curative procedure—surgical resection of the epileptogenic zone (EZ), which can be defined as the minimal area of brain tissue responsible for generating the recurrent seizure activity (12). However, to be effective, this procedure depends on correct identification of the EZ, which is often unclear. A comprehensive pre-surgical evaluation is necessary to pinpoint the EZ as well as to identify the risk of neurologic morbidity such as visual or speech impairment. Various non-invasive and invasive methods are used. Non-invasive techniques include scalp EEG and video-EEG monitoring, neuropsychological tests, speech-language studies, and brain imaging (magnetic resonance imaging (MRI), positron emission tomography (PET), ictal single-photon emission computed tomography (SPECT)). Of these methods, the highest predictor of surgical success is identification of a single visible MRI lesion (13, 14).

In patients with non-lesional MRI, localization and surgical success in seizure control are even more challenging. When the non-invasive methods of localization fail to identify the EZ, the method of last resort is an invasive evaluation, comprising the implantation of subdural grid electrodes (SDE) through open craniotomies, or depth electrodes through stereotactic EEG (SEEG). The process of identifying the EZ then involves visually inspecting tens to hundreds of invasive EEG signals without much assistance from computational tools. Currently, epileptologists study the onset of seizure events that occur over several days. Seizure events are typically marked by the early presence of beta-band activity (‘beta buzz’) or bursts of high frequency oscillations (100-300 Hz), which typically occur milliseconds before the clinical onset of seizures (15). Assuming the epileptic cortex generates epileptiform activity, which then entrains other regions into a clinical seizure (15), channels where these onset features first appear are commonly identified as the epileptogenic zone. Electrodecremental responses (loss of rhythmic activity) are also often observed. In general, epileptologists look at a variety of signatures to make their decision (15). Despite all of these possible EEG signatures, definition of the EZ may remain unclear for non-lesional patients. There thus remains a need for improved systems and methods for evaluating epileptic zones of a subject's brain.

### SUMMARY

According to some embodiments of the invention, a method of identifying an epileptogenic zone of a subject's brain is provided. The method includes receiving a plurality N of electrical signals that extend over a seizure duration from a corresponding plurality N of surgically implanted electrodes in the subject's brain, calculating within a time window components of an N×N adjacency matrix between each pair of the plurality of surgically implanted electrodes based on at least a portion of each of the plurality N of electrical signals, calculating N eigenvectors from the N×N adjacency matrix, and selecting an eigenvector of the N eigenvectors having a largest eigenvalue. The method further includes assigning an integer rank from 1 to N to each component of the selected eigenvector to provide an N×1 rank vector corresponding to the time window, sliding the time window by a time increment and repeating the immediately preceding three steps for the incremented time window, and repeating the immediately preceding step a plurality of times to provide the rank vector for a plurality of times, wherein each component of the rank vector corresponds to one of the N electrodes thus providing a rank signal for each of the N electrodes. The method further includes normalizing each rank signal by the number of electrodes N and the seizure duration to provide corresponding normalized signals, extracting a multidimensional feature vector from each normalized signal to provide a plurality N of multidimensional feature vectors, projecting each of the plurality N of multidimensional feature vectors onto a reduced dimensionality space, and receiving a plurality of training data points represented in the reduced dimensionality space, each of the plurality of training data points containing information of an electrode node regarding whether brain tissue corresponding to the electrode node was resected or non-resected brain tissue and whether a corresponding surgery was successful. The method further includes calculating grid weights for each feature vector projected into the reduced dimensionality space using the training data points, and assigning a numerical value to each surgically implanted electrode using the grid weights as an indication of whether the corresponding surgically implanted electrode is in an epileptogenic zone of the subject's brain.

According to some embodiments of the invention, a non-transitory computer-readable medium for identifying an epileptogenic zone of a subject's brain includes computer-executable code that, when executed by a computer causes the computer to perform the above-noted methods.

According to some embodiments of the invention, a system for identifying an epileptogenic zone of a subject's brain includes a computer configured to perform the above-noted methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Further objectives and advantages will become apparent from a consideration of the description, drawings, and examples.

FIG. 1 illustrates the process of computing a connectivity matrix for each pair of nodes;

FIG. 2 illustrates the eigenvalue centrality interpretation;

FIG. 3 shows normalization of the Y-axis of the rank evolution signals;

FIG. 4 shows normalization of the X-axis of the rank evolution signals;

FIG. 5 illustrates normalization of the rank evolution signals such that each signal integrates to 1;

FIG. 6 illustrates the extraction of the locations in time at which the signal integrates to 10% of the total, which form a 10×1 feature vector for the signal;

FIG. 7 shows an example two-dimensional principal component space with labeled electrodes plotted therein;

FIG. 8 shows a plot of the mean rank signature of points in each partition for each of the four labeled partitions in FIG. 7;

FIG. 9 illustrates how weights can be computed from data samples according to some embodiments of the invention;

FIG. 10 shows a weighting function in 2D principal component space for temporal lobe iEEG patients;

FIG. 11 shows a weighting function in 2D principal component space for occipital lobe iEEG patients;

FIG. 12 shows a weighting function in 2D principal component space for temporal lobe SEEG patients;

FIG. 13 shows a weighting function in 2D principal component space for occipital lobe SEEG patients.

FIG. 14 summarizes the analysis of a patient's EEG signals in preparation for creating a heat map;

FIG. 15 illustrate the process of assigning a numerical value or color to each electrode on a patient's brain; and

FIG. 16 illustrates an exemplary heat map wherein each electrode is assigned a color indicating a likelihood that the electrode is in the EZ.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Recently, we showed that intracranial electroencephalograms (EEGs) and stereoelectroencephalographs (SEEGs) are rich in information beyond the typical signatures clinicians use to identify the epileptic zone (EZ) (16-20). In particular, by viewing the epileptic brain as a dynamic networked system where EEG signals are correlated both temporally and spatially, we constructed a set of network-based statistics whose temporal evolution distinguishes the epileptic nodes from the non-epileptic nodes within specific epileptic networks, i.e., an electrophysiological signature of the EZ (19, 20). The electrophysiologic signature of the EZ has an “arch” shape. Networked-based analysis does not assume that recordings from different EEG channels are independent, but instead assumes that they are samples of activity from brain regions that are structurally and/or functionally connected and are therefore dependent (16-18).

According to some embodiments of the invention, we use time series network-based statistics and the identified EZ “arch” signature to develop a tool, EZTrack, that takes as input intracranial EEG data and the patient's brain image after electrode implantation, and outputs a “heat map” which colors an electrode red if the likelihood of it being in the EZ is high and blue if the likelihood is low. Intermediate colors are used for intermediate likelihoods. The colors “red” and “blue” are purely exemplary, and other colors or other means of identifying and correlating electrodes with different likelihoods can also be used.

An embodiment of the current invention provides a method of identifying an epileptogenic zone of a subject's brain. The method includes receiving a plurality N of electrical signals that extend over a seizure duration from a corresponding plurality N of surgically implanted electrodes in the subject's brain, calculating within a time window components of an N×N adjacency matrix between each pair of the plurality of surgically implanted electrodes based on at least a portion of each of the plurality N of electrical signals, calculating N eigenvectors from the N×N adjacency matrix, and selecting an eigenvector of the N eigenvectors having a largest eigenvalue. The method further includes assigning an integer rank from 1 to N to each component of the selected eigenvector to provide an N×1 rank vector corresponding to the time window, sliding the time window by a time increment and repeating the immediately preceding three steps for the incremented time window, and repeating the immediately preceding step a plurality of times to provide the rank vector for a plurality of times, wherein each component of the rank vector corresponds to one of the N electrodes thus providing a rank signal for each of the N electrodes. The method further includes normalizing each rank signal by the number of electrodes N and the seizure duration to provide corresponding normalized signals, extracting a multidimensional feature vector from each normalized signal to provide a plurality N of multidimensional feature vectors, projecting each of the plurality N of multidimensional feature vectors onto a reduced dimensionality space, and receiving a plurality of training data points represented in the reduced dimensionality space, each of the plurality of training data points containing information of an electrode node regarding whether brain tissue corresponding to the electrode node was resected or non-resected brain tissue and whether a corresponding surgery was successful. The method further includes calculating grid weights for each feature vector projected into the reduced dimensionality space using the training data points, and assigning a numerical value to each surgically implanted electrode using the grid weights as an indication of whether the corresponding surgically implanted electrode is in an epileptogenic zone of the subject's brain.

According to some embodiments of the invention, the method further includes displaying a heat map based on the numerical values assigned to each surgically implanted electrode superimposed on a rendering of the subject's brain and corresponding plurality N of surgically implanted electrodes. A variety of different methods can be used for reducing the dimensionality of the feature space. According to some embodiments, principal component analysis (PCA) is employed. However, the embodiments of the invention are not limited to PCA, and other reduction methods may be used. According to some embodiments of the invention, the reduced dimensionality space can be a smaller dimension space than a dimensionality of the multidimensional feature vectors. For example, the multidimensional feature vectors can be ten-dimensional feature vectors and the principal component space can be a two-dimensional space. Calculating components of the N×N adjacency matrix between each pair of the plurality of surgically implanted electrodes can use the formula

Aᵢⱼ(t)=[∫_{30 Hz}^{90 Hz}Pᵢ(f)Pⱼ(f)df](t)

where Pᵢ(f) and Pⱼ(f) are magnitudes of Fourier transforms of the portion of the electrical signal corresponding to the first time period from electrodes i and j, respectively, of the plurality of electrodes. However, the general concepts of the current invention are not limited to connectivities calculated as in the equation above. Further, different integration limits can be selected in other embodiments of the current invention.

According to some embodiments of the invention, calculating grid weights comprises calculating a weighting function in the reduced feature space (e.g., a PC space like the one defined above). The user can define the weighting function. Two possible weighting functions are described herein, (i) Bayesian weights derived from data and (ii) Gaussian mixtures. However, the embodiments of the invention are not limited to these types of weighting function, and other weighting functions can be used. Extracting the multidimensional feature vector can include assigning each component of the multidimensional feature vector a value equal to an area from zero to each successive tenth interval under a corresponding rank signal curve.

A non-transitory computer-readable medium for identifying an epileptogenic zone of a subject's brain according to some embodiments of the current invention includes computer-executable code that, when executed by a computer causes the computer to perform the above-noted methods. A system for identifying an epileptogenic zone of a subject's brain according to some embodiments of the current invention includes a computer configured to perform the above-noted methods.

Further additional concepts and embodiments of the current invention will be described by way of the following examples. However, the broad concepts of the current invention are not limited to these particular examples.

### EXAMPLES

According to some embodiments of the invention, data for patients that had resective surgeries was analyzed to provide information for patience considering a resective surgery. Data was obtained for 42 patients that had resective surgeries. 20 patients were implanted with SDE and 22 with SEEG. EEG data on 1-3 seizures was analyzed by according to the methods described herein without knowledge of the surgical outcomes. In 40 out of 42 situations, we were able to predict successful surgical outcomes due to overlap between EZTrack's heatmaps and the actual area of resection. Compellingly, in all 17 failures, we were able to predict negative surgical outcomes due to the lack of overlap between EZTrack's heatmaps and the resected areas. These findings suggest that, had clinicians used EZTrack to assist in localizing the EN in these patients, they may have either resected different regions or refrained from performing the surgeries.

Such an assistive tool would not only reduce extra-operative monitoring time in the EMU, thereby cutting medical costs and decreasing complications associated with invasive monitoring, but would also increase seizure freedom rates, especially in the more difficult to localize patients (i.e. non-lesional patients).

In the description below, data for two types of EEG patients, SEEG and EcoG, is analyzed. However, the embodiments of the invention are not limited to these two types of data. The methods and algorithms described herein can be applied to many different types of data, including, for example, all types of EEG data.

Stereotactic EEF Monitoring Technique—SEEG

The Cleveland Clinic is a world-renowned center for the evaluation and treatment of epilepsy, assessing around 9,500 patients every year from all 50 states and more than 10 countries. More than 400 associated epilepsy surgeries are performed every year, including a growing number of stereotactically placed EEG (SEEG) electrodes (21-23), a technique that was developed in France, and brought to the Unites States by Dr. Jorge Gonzalez-Martinez.

In routine placement of depth electrodes, burr-holes that are each 15 mm in diameter are required for safe visualization of cortical vessels, and therefore only a small number of electrodes are placed. SEEG placement, however, uses several small drill holes (1.8 mm in diameter), allowing many electrodes to be inserted (up to 20). SEEG provides a more complete coverage of the brain, from lateral, intermediate, and/or deep structures in a three-dimensional arrangement recorded over hundreds of channels.

Since direct visualization of the cortical surface is not possible with small drills, the SEEG technique requires detailed pre-procedural vascular mapping using pre-operative imaging with magnetic resonance angiography (MRA) and Computerized Tomography Angiograms (CTA). The mapping procedure is performed under fluoroscopy using general anesthesia, and an expert neuro-anesthesiologist correctly titrates anesthesia to permit measurement of intracranial EEG. The number and location of implanted electrodes are pre-operatively planned based on a pre-implantation hypothesis, which is formulated in accordance with non-invasive pre-implantation data such as seizure semiology, ictal and inter-ictal scalp EEG, MRI images, PET, and ictal SPECT scans. Thus, the implantation strategy has the goal of accepting or rejecting the pre-implantation hypothesis of the location of the EZ. During implantation, a medical expert views a 3D overlay of the pre-op image and the angiography and places the electrodes in paths that do not intersect any vessels. Despite the advantages of SEEG over SDE, SEEG recordings are as difficult to analyze as SDE recordings through visual inspection, requiring an assistive tool to localize the EZ.

Intracranial EEG Monitoring Technique—EcoG

Intracranial ECoG recordings were acquired through subdural grid arrays, subdural strips, or depth-electrode arrays in various combinations as determined by clinical assessment. Subdural grids have 20-64 contacts per array and were used in combination with subdural strips (4-8 contacts) or depth arrays, thus having 80-116 recording electrodes per patient over all. Intracranial contact locations were documented by post-operative CT co-registration with MRI. Signals were acquired using continuous multi-channel ECoG recordings collected over 5 days on average (min.: 2 days; max: 10 days), which led to 130.8±40.9 hours of consecutive recordings per patient (mean±S.D.). Recordings were acquired with a Stellate™ system (Stellate Systems, Inc., Montreal, QC) with 1000 Hz sampling rate and 300 Hz anti-aliasing filter, and were converted to EDF format for storage and further processing. Each EDF file stores approximately 42 minutes of continuous ECoG data from all the channels and it is automatically generated by the acquisition system. Consecutive EDF files cover consecutive, non-overlapping, time windows with less than 5s-lag in between. Digitized data were stored in a IRB-approved database compliant with HIPAA (Health Insurance Portability and Accountability Act) regulations.

Board-certified electroencephalographers (up to three) marked, by consensus, the unequivocal electrographic onset of each seizure and the period between seizure onset and termination. The seizure onset was indicated by a variety of stereotypical electrographic features, which include, but were not limited to, the onset of fast rhythmic activity, an isolated spike or spike and wave complex followed by rhythmic activity, or an electrodecremental response (7). Concurrently with the examination of the ECoG signals, changes in the patient's behavior were also sought from the video segment of video-EEG recordings.

For each patient, we combined (i) surgical notes of the electrodes corresponding to the resected regions of the brain and (ii) postoperative follow-up information describing how the resection affected the patient's seizures. If, after surgery, a patient reported no seizures or could manage epilepsy with medication, then the resected areas were determined to correspond to the epileptogenic region (focus). If the patient continued to have seizures after the resection, the resected region was determined to be smaller than or outside of the epileptogenic region. Finally, if the patient required multiple resective surgeries, the resected areas were determined to be larger than the seizure focus.

Network-Based Data Analytics

Our raw dataset consisted of EEG recordings of seizures with 60 seconds of data before and after each seizure. Data was collected from 42 patients (22 SEEG, 20 ECoG) with at least two seizures per patient. However, the embodiments of the invention are not limited to datasets having the characteristics described above. For example, dataset comprising more or less time before or after a seizure can also be used. We applied an algorithm to the data that consists of steps that can be applied to any EEG data type. According to some embodiments, an algorithm is applied that considers each electrode in the EEG array to be a node in a network. Note that EZTrack was trained and tested on SEEG and ECoG cohorts separately, however the data processing steps, enumerated below, are identica, and can be applied to other types of EEG data as well.

The following describes computational steps according to some embodiments of the current invention. The computational steps can be used to calculate weighting functions that indicate whether an electrode in a patient's brain is in an epileptogenic zone. Many of the computational steps can then be repeated and the weighting function can be applied to a patient's EEG recording to create a heat map indicating which electrodes in the patient's brain are likely in the epileptogenic zone, and which are not.

Compute and Rank Nodal Centrality Over Time

Network centrality for each node in each of the patients was computed every second using a 2.5 second sliding window sliding every second 60 seconds before seizure, during seizure, and 60 seconds after seizure for two seizure events. The length of the window is exemplary, and other time periods can also be used. For each window, the brain network was represented by a connectivity matrix (24). Any dependency matrix can be used, and the embodiments of the invention are not limited to the type of dependency matrix or the frequency band disclosed herein. According to some embodiments, a connectivity matrix is calculated by computing all pairwise cross-power in the gamma frequency band (30-90 Hz), i.e.,

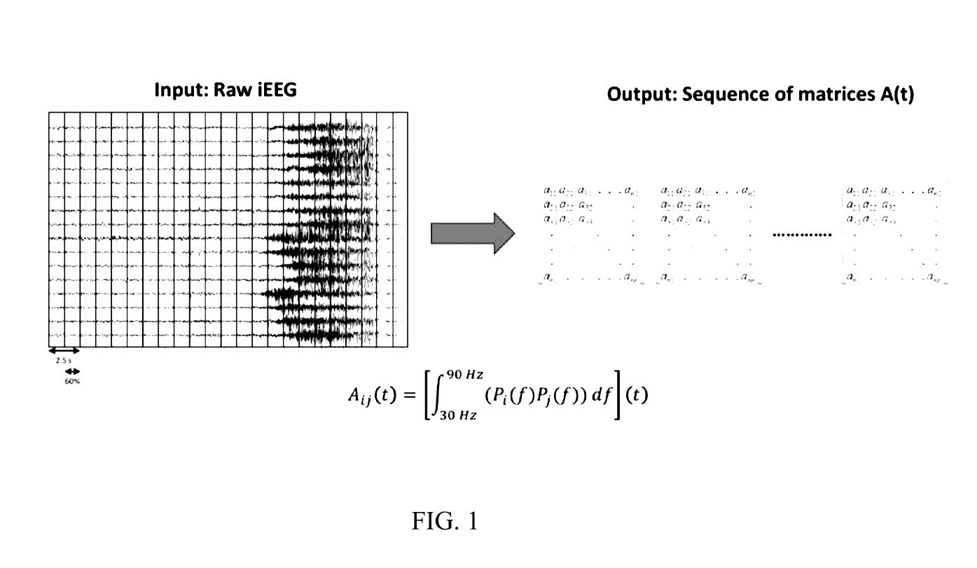

Aᵢⱼ=∫_{30 Hz}^{90 Hz}(Pᵢ(f)Pⱼ(f))df

where Pᵢ, Pⱼ are the magnitudes of the Fourier transform of the time series in the window recorded from electrodes i and j respectively. The process is illustrated in FIG. 1. The gamma frequency band often exhibited the most modulation in power between non-seizure and seizure periods and has been thought to be correlated to neuronal spiking and fMRI activity and thus carries information in such invasive recordings (25-27). However, the general concepts of the current invention are not limited to connectivities calculated as in the equation above. Further, different integration limits can be selected in other embodiments of the current invention.

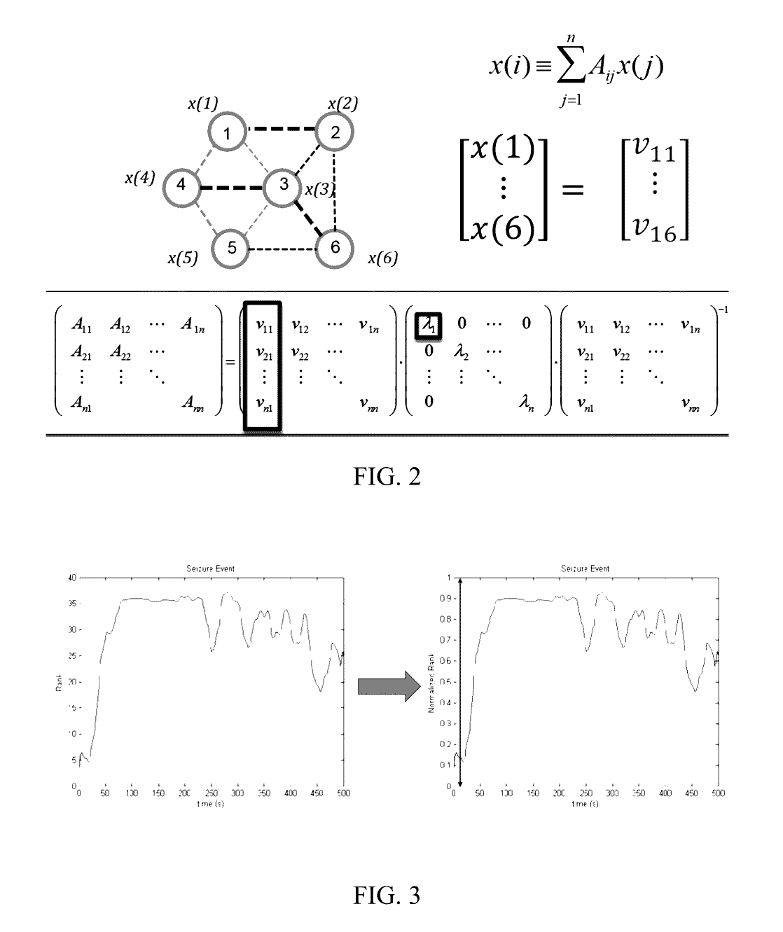

The importance of each electrode to the network connectivity was measured by the strength and number of connections it makes with other electrodes, referred to as centrality. Any definition of centrality can be used, including but not limited to eigenvector centrality (EVC), degree, page rank, etc. According to some embodiments, EVC is used to measure the connectivity of each electrode. However, the embodiments of the invention are not limited to EVC, and other types of centrality can also be used. FIG. 2 illustrates the EVC interpretation. The EVC of an electrode is defined as the sum of the EVCs of all other electrodes weighted by their connectivity. The EVC of all electrodes is computed implicitly as:

EVC(i)=λΣⱼ₌₁^{N}AᵢⱼEVC(j).

λ is the leading eigenvalue of Aᵢⱼ and the EVC is then the leading eigenvector of Aᵢⱼ. In simple terms, the EVC of a node in the network (electrode) is proportional to the sum of EVCs of its neighbors (nodes it is connected to). That is, a node is important if it is (i) connected to a few nodes that are themselves very important or if it is (ii) connected to a very large number of not-so-important nodes.

The leading eigenvectors of connectivity matrices were calculated numerically at each second during the recordings from the connectivity matrices. Finally, the EVC vector for each second was converted to a ranked vector containing values 1 to N, where a 1 was placed in the component of EVC that had the largest centrality and an N was placed in the component of EVC that had the smallest centrality.

Normalize the Rank Evolution Signals

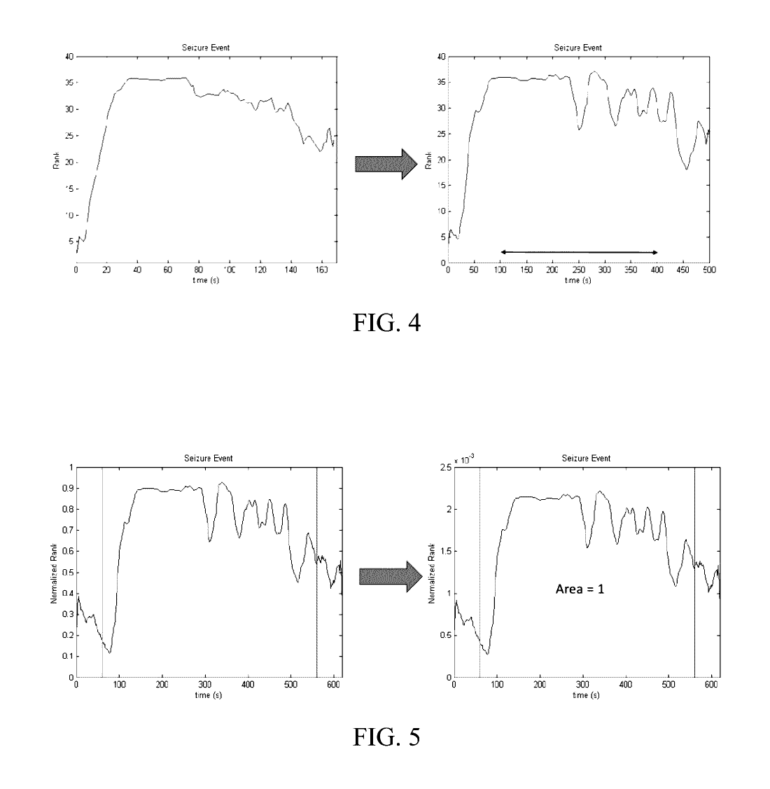

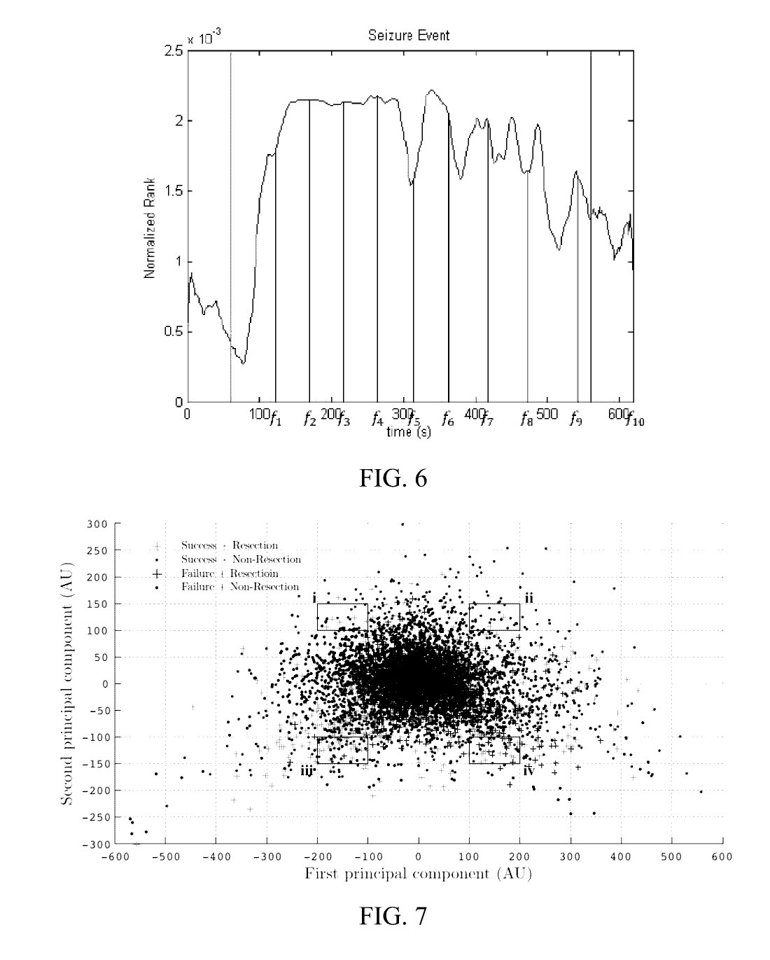

Next we normalized the rank evolution signals in the X and Y directions. To normalize the Y-axis, we divided the ranking of each electrode by N, as shown in FIG. 3. This was done so that we could compare signals from different patients that have varying numbers of electrodes. To normalize the X-axis, we normalized each signal such that all signals were 500 seconds in length, as shown in FIG. 4. Most signals were under 500 seconds in length, so the majority of the signals were stretched. This was done so that the shape of the signals could be compared, and so that the range of two-dimensional (2D) principal component space described below would be invariant. In order to compare the signals in a quantifiable manner, we normalized all signals once more such that the signal integrates to 1, as shown in FIG. 5. This normalization converted each signal into a probability density function, although we do not use the properties associated with such functions. For each normalized signal, we extract the locations in time at which the signal integrates to 10% of the total, i.e., points in normalized time where the signal integrates to 0.1, 0.2, 0.3, and so on until the end of the signal is reached. This process is shown in FIG. 6. This gives a 10×1 vector for each signal that serves as a feature vector for that signal.

Perform Principal Component Analysis

Once we calculated feature vectors for each signal, we projected the features into a reduced dimensionality space. According to some embodiments, principal component analysis (PCA) is employed. However, the embodiments of the invention are not limited to PCA, and other reduction methods may be used. According to some embodiments of the invention, the reduced dimensionality space can be a smaller dimension space than a dimensionality of the multidimensional feature vectors. According to some embodiments, once we calculated feature vectors for each signal, we projected the features into a 2D principal component space by performing principal component analysis and plotting the first and second principal components. We then placed a grid on the 2D principal component plot and compared signatures within each square. Each electrode was labeled according to whether or not the electrode was resected and whether the resection was a success or a failure, producing four categories of labels. An example two-dimensional principal component space with labeled electrodes plotted therein is shown in FIG. 7. We created a weighting function and separated the patients into subsets according to the lobe in which the majority of the electrodes were located.

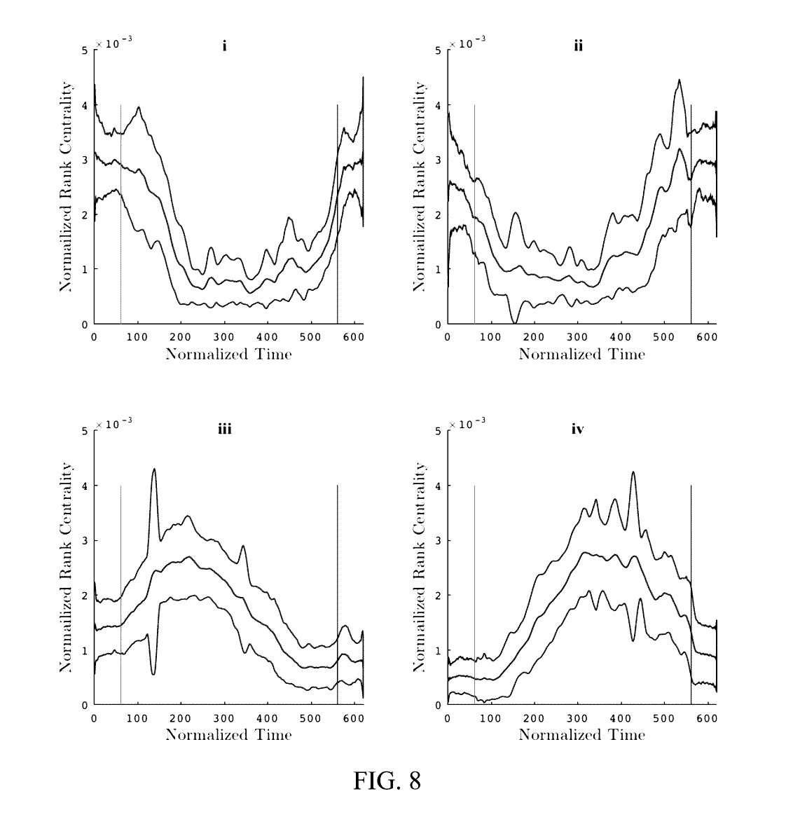

In order to visualize the rank signature as a function of 2D coordinates in 2D principal component space, the space was discretized by equisized partitions. A 2D distribution of rank signature was then obtained by plotting mean rank signature of points in each partition. This is illustrated in FIG. 8, where the four plots in FIG. 8 correspond to the four labeled partitions in FIG. 7. To capture the variability of mean rank signature in space as the corresponding likelihood in successful resection (or a failure non-resection) of an electrode we defined weighting functions as bivariate Gaussians. Essentially, we observed that the space can be subdivided into four quadrants with each quadrant having different parameters (exponential decay factor and covariance matrix) for the bivariate Gaussian. Mean vector or the center for Gaussian was varied as a function of lobe (temporal or occipital) and data type (iEEG or SEEG). A covariance matrix was first obtained from points in all four categories and then parameterized for each quadrant. Variance along each axes was changed but covariance (ellipticity) was invariant across all quadrants. Similarly, the decay constant in the exponential was parameterized for each quadrant. The overall weighting function is defined as the linear combination of four Gaussians multiplied by appropriate Heaviside step functions.

$$w ( x , y ) {{\sum }^{4}}_{i = 1} {h}_{i} ( x , y ) {w}_{i} ( x , y )$$

$${w}_{i} ( x , y ) = {e}^{\{ - {{α}_{i} [ \begin{matrix}x - {μ}_{x} \\ y - {μ}_{y}\end{matrix} ]}^{T} {\sum }_{i}^{- 1} [ \begin{matrix}X - {μ}_{x} \\ y - {μ}_{y}\end{matrix} ] \}}$$

αᵢ—exponential decay factor for i^{th} quadrant
   Σᵢ—covariance matrix of i^{th} quadrant

hᵢ(x, y)=Θ(x−μₓ)Θ(y−μ_{y})
(x,y)∈ i^{th} quadrant

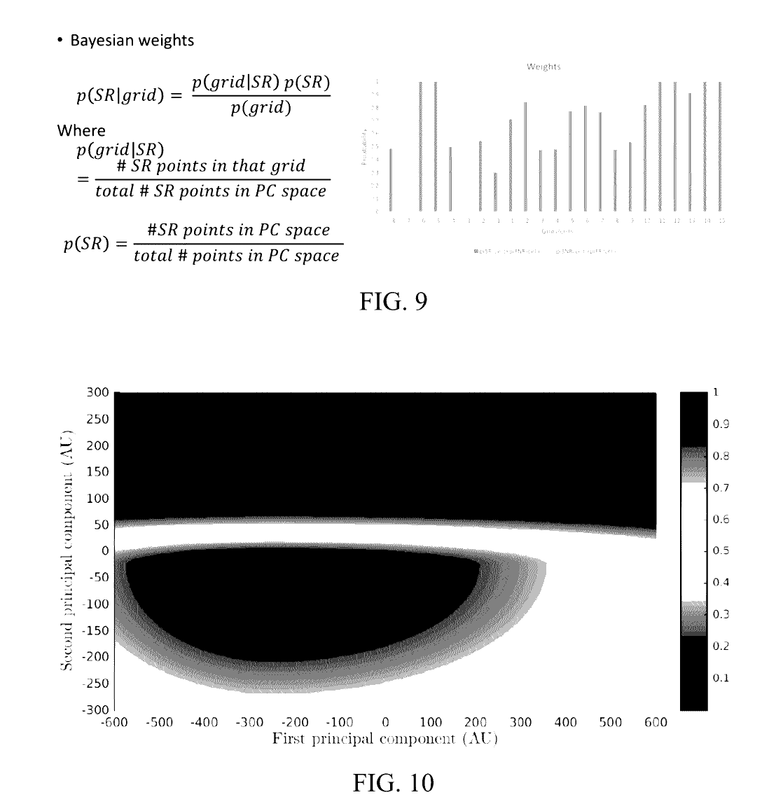

For each subset, we would remove one patient and train the weighting function of the remaining patients, then test the accuracy of the function on the patient that was removed from the set. The weighting function was parameterized by the center such that only the center could change. The center was changed multiple times and the accuracy was tests on all patients in the subset. According to some embodiments of the invention, the weights can be defined using the equations shown in FIG. 9. FIG. 9 is an illustration of how the weights can be computed from data samples alone. A grid is defined as a rectangular section in the PC space. SR corresponds to an electrode implanted in a patient who had a successful surgical outcome (S) and whose brain area near the electrode was resected (R). According to some embodiments, the weights are determined based on the Gaussian mixtures defined above. According to some embodiments, the node weights are calculated according to the following formula:

$$Node weight = \frac{{\sum }_{1}^{n} [ {p}_{i} ( grid \vert SR ) + {p}_{i} ( grid \vert FNR ) ]}{n}$$

where n is the number of electrode samples in the given grid. However, the embodiments of the invention are not limited to these types of weighting function, and other weighting functions can be used.

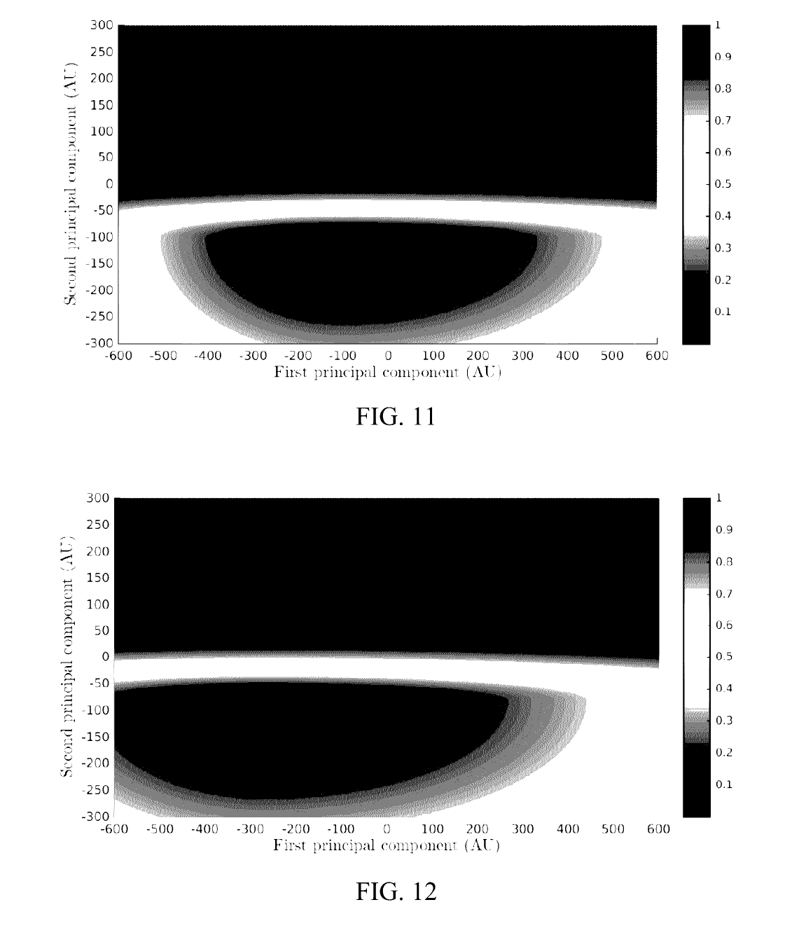

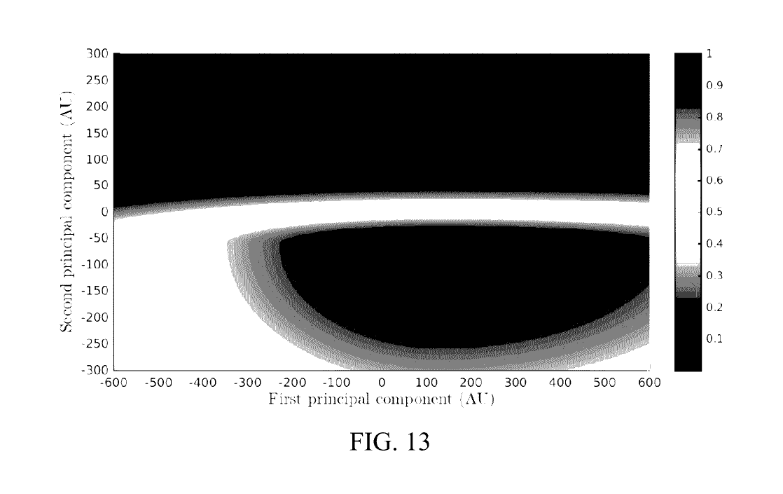

Example weighting functions for each quadrant are shown in FIGS. 10-13. FIG. 10 shows a weighting function in 2D principal component space for temporal lobe iEEG patients. FIG. 11 shows a weighting function in 2D principal component space for occipital lobe iEEG patients. FIG. 12 shows a weighting function in 2D principal component space for temporal lobe SEEG patients. FIG. 13 shows a weighting function in 2D principal component space for occipital lobe SEEG patients.

Pre-Surgical Evaluation

According to some embodiments of the invention, the calculated weighting function can be used to determine whether an electrode in a patient's brain is in the EZ. A heat map can be created to indicate the likelihood that each electrode is in the EZ. Accordingly to some embodiments, EEG recordings from N nodes in the patient's brain during a seizer are obtained. According to some embodiments, the recordings also include 60 second of data before and after the seizure.

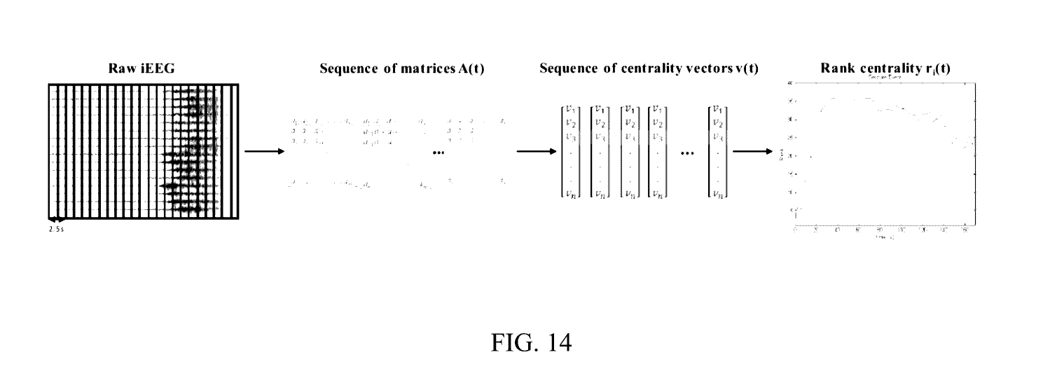

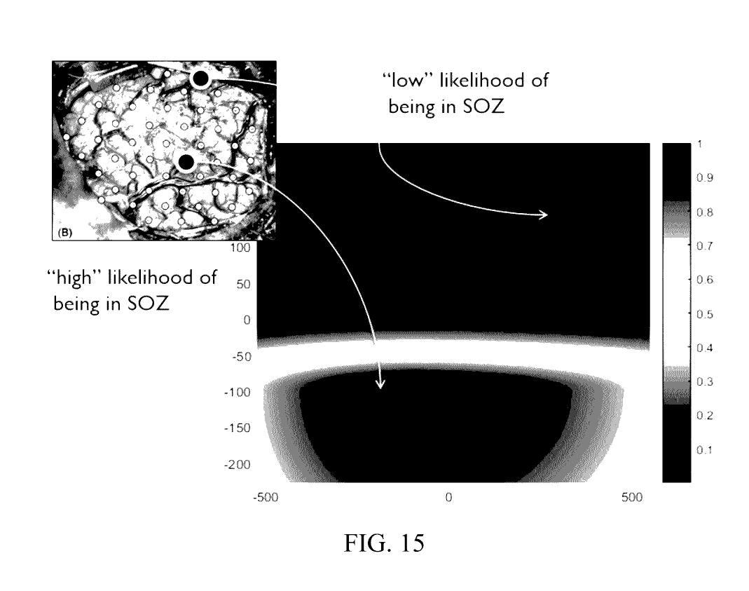

Many of the computational steps outlined above are then repeated for the patient's EEG data, as summarized in FIG. 14. Network centrality for each node is computed every second using a 2.5 second sliding window sliding every second 60 seconds before seizure, during seizure, and 60 seconds after seizure for the seizure event. The length of the window is exemplary, and other time periods can also be used.

Eigenvector centrality is then used to measure the connectivity of each electrode. The leading eigenvectors of connectivity matrices are calculated numerically at each second during the recording from the connectivity matrices. Finally, the EVC vector for each second is converted to a ranked vector containing values 1 to N, where a 1 is placed in the component of EVC that has the largest centrality and an N is placed in the component of EVC that has the smallest centrality.

Next, the rank evolution signals are normalized in the X and Y directions, as described in detail above. The signals are also normalized such that each signal integrates to 1. For each normalized signal, we extract the locations in time at which the signal integrates to 10% of the total, i.e. points in normalized time where the signal integrates to 0.1, 0.2, 0.3, and so on until the end of the signal is reached. This gives a 10×1 vector for each signal that serves as a feature vector for the signal.

Once we calculate feature vectors for each signal, we project the features into a reduced dimensionality space. According to some embodiments, PCA is employed. However, the embodiments of the invention are not limited to PCA, and other reduction methods may be used. According to some embodiments of the invention, the reduced dimensionality space can be a smaller dimension space than a dimensionality of the multidimensional feature vectors. According to some embodiments, once we calculate feature vectors for each signal, we project the features into two-dimensional principal component space by performing principal component analysis and plotting the first and second principal components. We then apply the appropriate weighting function (SEEG or ECoG, temporal or occipital) to the two-dimensional projection, and assign a value to each electrode indicating the likelihood that electrode is in the EZ, as show in FIG. 15. For example, if the patient has SEEG electrodes that are primarily located in the occipital lobe, a weighting function based on patient data for patients having SEEG electrodes primarily located in the occipital lobe can be used. An example of this weighting function is shown in FIG. 13. The weighting function assigns a value to every point in the two-dimensional principal component space, and thus a value can be assigned to each electrode based on the first and second principal components corresponding to that electrode.

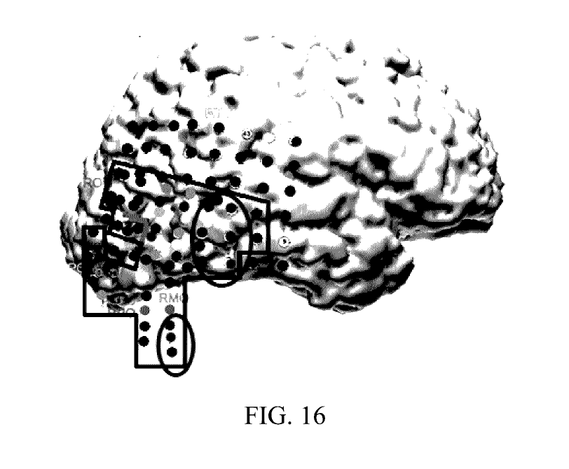

According to some embodiments of the invention, the numerical value for each of the patient's N electrodes can be represented by a color, and the N colored electrodes can be combined to form a heat map. An exemplary heat map is shown in FIG. 16. A user can use the heat map to determine which electrodes are in the EZ. This can inform a decision regarding resection, as the heat map may indicate whether a resection of an area corresponding to a particular electrode or group of electrodes would potentially reduce or eliminate seizure activity.

According to some embodiments, the weighting functions can be continually updated as more data regarding surgical resections is collected.

### REFERENCES

[1] Brodie, M. J., Shorvon, S. D., Canger, R. et al. Commission on European Affairs: appropriate standards of epilepsy care across Europe. Epilepsia 1997; 28:1245-1250.

[2] Berg A T, Kelly M M. Defining intractability: comparisons among published definitions. Epilepsia. 2006 February; 47(2):431-6.

[3] Kwan P, Brodie M J. Early identification of refractory epilepsy. N Engl J Med. 2000 Feb. 3; 342(5):314-9.

[4] Berg, A T. Identification of Pharmacoresistant Epilepsy. Neurol Clin 27 (2009) 1003-1013.

[5] Murray M I, Halpern M T, Leppik I E. Cost of refractory epilepsy in adults in the USA. Epilepsy Research. 1996; 23:139-148.

[6] Begley C E, Famulari M, Annegers J F, Lairson D R, Reynolds T F, Coan S, Dubinsky S, Newmark M E, Leibson C, So E L, Rocca W A. The cost of epilepsy in the United States: an estimate from population-based clinical and survey data. Epilepsia 2000; 41: 342-351.

[7] Ferro, M A, Speechley K N. Depressive symptoms among mothers of children with epilepsy: a review of prevalence, associated factors, and impact on children. Epilepsia 2009; 50(11): 2344-2354.

[8] Schuele S U, Lüders H O. Intractable epilepsy: management and therapeutic alternatives. Lancet Neurol. 2008 June; 7(6):514-24.

[9] Hermann B P, Seidenberg M, Dow C, Jones J, Rutecki P, Bhattacharya A, Bell B. Cognitive prognosis in chronic temporal lobe epilepsy. Ann Neurol. 2006 July; 60(1):80-7.

[10] Gilliam F G, Kuzniecky R, Meador K. Patient-oriented outcome assessment after temporal lobectomy for refractory epilepsy. Neurology 1999; 53:687-94.

[11] Gilliam F G. Diagnosis and treatment of mood disorders in persons with epilepsy. Curr Opin Neurol. 2005 April; 18(2):129-33.

[12] Lüders H O, Najm I, Nair D, Widdess-Walsh P, Bingman W. The epileptogenic zone: general principals. Epileptic Disord. 2006 August; 8 Suppl 2:S1-9. Erratum in: Epileptic Disord. 2008 June; 10(2):191.

[13] McIntosh A M, Kalnins R M, Mitchell L A, Fabinyi G C, Briellmann R S, Berkovic S F. Temporal lobectomy: long-term seizure outcome, late recurrence and risks for seizure recurrence. Brain. 2004 September; 127(Pt 9):2018-30. Epub 2004 Jun. 23.

[14] Jeha L E, Najm I M, Bingaman W E, Khandwala F, Widdess-Walsh P, Morris H H, Dinner D S, Nair D, Foldvary-Schaeffer N, Prayson R A, Comair Y, O'Brien R, Bulacio J, Gupta A, Lüders H O. Predictors of outcome after temporal lobectomy for the treatment of intractable epilepsy. Neurology. 2006 Jun. 27; 66(12):1938-40.

[15] Fisher R S. “Therapeutic devices for epilepsy,” Ann Neurol. 2012; 71:157-68.

[16] Santaniello S, Burns S P, Golby J, Singer J, Anderson W S, Sarma S V. (2011) Quickest Detection of Seizure Onsets in Drug-Resistant Patients: An Optimal Control Approach. Epilepsy &. Behavior. 22, pp 49-60.

[17] Kerr M, Burns S, Gale J, Sarma S V (2011) Multivariate Analysis of SEEG Signals During Seizure. Proceedings of the 33rd IEEE EMBS Conference.

[18] Yaffe R, Burns S, Gale J, Park H J, Bulacio J, Gonzalez-Martinez J, Sarma S V. Brain State Evolution During Seizure and under Anesthesia: A Network-Based Analysis of Stereotaxic EEG Activity in Drug-Resistant Epilepsy Patients. Proceedings of the 34th IEEE EMBS Conference.

[19] Sarma S V, Subramanian S., Hao S. Computational tool for pre-surgical evaluation of patients with medically refractory epilepsy. Patent (filed October 2012)

[20] Subramanian S, Hao S, Santaniello S, Yaffe R, Burns S P, Bergey G, Jouny C, Crone N, Anderson W S, Sarma SV (Under Review). Identifying Intracranial EEG Signatures of the Epileptogenic Zone Using Network Centrality. Journal of Computational Neuroscience.

[21] Najm I, Bingaman W, Lüders H. (2002) The use of subdural grids in the management of focal malformations due to abnormal cortical develop-ment. Neurosurg Clin N Am 13:87-92.

[22] Bancaud J, Angelergues R, Bernouilli C, Bonis A, Bordas-Ferrer M, Bresson M, Buser P, Covello L, Morel P, Szikla G, Takeda A, Talairach J. (1970) Functional stereotaxic exploration (SEEG) of epilepsy. Electro-encephalogr Clin Neurophysiol 28:85-86.

[23] Kahane P, Minotti L, Hoffmann D, Lachaux J P, Ryvlin P. (2004) Invasive EEG in the definition of the seizure onset zone: depth electrodes. In: Rosenow F, Lüders H (Eds) Presurgical assessment of the epilepsies with clinical neurophysiology and functional imaging. Elsevier, Amsterdam, the Netherlands, pp. 109-133.

[24] Newman M J (2010) Networks: An Introduction. 720 pgs. Oxford University Press, USA.

[25] Gotman J. “Measurement of small time differences between EEG channels: method and application to epileptic seizure propagation,” Electroenceph Clin Neurophysiol. 1983; 56(5):501-14.

[26] Wu L, Gotman J. “Segmentation and classification of EEG during epileptic seizures,” Electroenceph Clin Neurophysiol. 1998; 106(4):344-56.

[27] Worrell G A, Parish L, Cranstoun S D, Jonas R, Baltuch G, Litt B. “High-frequency oscillations and seizure generation in neocortical epilepsy,” Brain. 2004; 127:1496-506.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A method of identifying an epileptogenic zone of a subject's brain, comprising:
receiving a plurality N of electrical signals that extend over a seizure duration from a corresponding plurality N of surgically implanted electrodes in said subject's brain;
calculating within a time window components of an N×N adjacency matrix between each pair of said plurality of surgically implanted electrodes based on at least a portion of each of said plurality N of electrical signals;
calculating N eigenvectors from said N×N adjacency matrix;
selecting an eigenvector of the N eigenvectors having a largest eigenvalue;
assigning an integer rank from 1 to N to each component of the selected eigenvector to provide an N×1 rank vector corresponding to said time window;
sliding said time window by a time increment and repeating the immediately preceding three steps for the incremented time window;
repeating the immediately preceding step a plurality of times to provide said rank vector for a plurality of times, wherein each component of said rank vector corresponds to one of said N electrodes thus providing a rank signal for each of the N electrodes;
normalizing each rank signal by the number of electrodes N and said seizure duration to provide corresponding normalized signals;
extracting a multidimensional feature vector from each normalized signal to provide a plurality N of multidimensional feature vectors;
projecting each of said plurality N of multidimensional feature vectors onto a reduced dimensionality space;
receiving a plurality of training data points represented in said reduced dimensionality space, each of said plurality of training data points containing information of an electrode node regarding whether brain tissue corresponding to said electrode node was resected or non-resected brain tissue and whether a corresponding surgery was successful;
calculating grid weights for each feature vector projected into said reduced dimensionality space using said training data points; and
assigning a numerical value to each surgically implanted electrode using said grid weights as an indication of whether the corresponding surgically implanted electrode is in an epileptogenic zone of said subject's brain.

2. The method of claim 1, further comprising displaying a heat map based on said numerical values assigned to each surgically implanted electrode superimposed on a rendering of said subject's brain and corresponding plurality N of surgically implanted electrodes.

3. The method of claim 1, wherein said reduced dimensionality space is a principal component space.

4. The method of claim 1, wherein said reduced dimensionality space is a smaller dimension space than a dimensionality of said multidimensional feature vectors.

5. The method of claim 4, wherein said multidimensional feature vectors are ten-dimensional feature vectors and said reduced dimensionality space is a two-dimensional space.

6. The method of any one of claims 1, wherein said calculating components of said N×N adjacency matrix between each pair of said plurality of surgically implanted electrodes uses the formula
Aᵢⱼ(t)=[ƒ_{30 Hz}^{90 Hz}Pᵢ(f)Pⱼ(f)df](t)
where Pᵢ(f) and Pⱼ(f) are magnitudes of Fourier transforms of said portion of said electrical signal corresponding to said first time period from electrodes i and j, respectively, of said plurality of electrodes.

7. The method of any one of claims 1, wherein said calculating grid weights comprises calculating Bayesian grid weights.

8. The method of claim 5, wherein said extracting said multidimensional feature vector comprises assigning each component of said multidimensional feature vector a value equal to an area from zero to each successive tenth interval under a corresponding rank signal curve.

9. A non-transitory computer-readable medium for identifying an epileptogenic zone of a subject's brain comprising computer-executable code, said code when executed by a computer causes the computer to:
receive a plurality N of electrical signals that extend over a seizure duration from a corresponding plurality N of surgically implanted electrodes in said subject's brain;
calculate within a time window components of an N×N adjacency matrix between each pair of said plurality of surgically implanted electrodes based on at least a portion of each of said plurality N of electrical signals;
calculate N eigenvectors from said N×N adjacency matrix;
select an eigenvector of the N eigenvectors that has a largest eigenvalue;
assign an integer rank from 1 to N to each component of the selected eigenvector to provide an N×1 rank vector corresponding to said time window;
slide said time window by a time increment and repeate the immediately preceding three steps for the incremented time window;
repeat the immediately preceding step a plurality of times to provide said rank vector for a plurality of times, wherein each component of said rank vector corresponds to one of said N electrodes thus providing a rank signal for each of the N electrodes;
normalize each rank signal by the number of electrodes N and said seizure duration to provide corresponding normalized signals;
extract a multidimensional feature vector from each normalized signal to provide a plurality N of multidimensional feature vectors;
project each of said plurality N of multidimensional feature vectors onto a reduced dimensionality space;
receive a plurality of training data points represented in said reduced dimensionality space, each of said plurality of training data points containing information of an electrode node regarding whether brain tissue corresponding to said electrode node was resected or non-resected brain tissue and whether a corresponding surgery was successful;
calculate grid weights for each feature vector projected into said reduced dimensionality space using said training data points; and
assign a numerical value to each surgically implanted electrode using said grid weights as an indication of whether the corresponding surgically implanted electrode is in an epileptogenic zone of said subject's brain.

10. The non-transitory computer-readable medium of claim 9, further comprising displaying a heat map based on said numerical values assigned to each surgically implanted electrode superimposed on a rendering of said subject's brain and corresponding plurality N of surgically implanted electrodes.

11. The non-transitory computer-readable medium of claim 9, wherein said reduced dimensionality space is a smaller dimension space than a dimensionality of said multidimensional feature vectors.

12. The non-transitory computer-readable medium of claim 11, wherein said multidimensional feature vectors are ten-dimensional feature vectors and said reduced dimensionality space is a two-dimensional space.

13. The non-transitory computer-readable medium of any one of claims 9, wherein said calculating components of said N×N adjacency matrix between each pair of said plurality of surgically implanted electrodes uses the formula
Aᵢⱼ(t)=[∫_{30 Hz}^{90 Hz}Pᵢ(f)Pⱼ(f)df](t)
where Pᵢ(f) and Pⱼ(f) are magnitudes of Fourier transforms of said portion of said electrical signal corresponding to said first time period from electrodes i and j, respectively, of said plurality of electrodes.

14. The non-transitory computer-readable medium of any one of claims 9, wherein said calculating grid weights comprises calculating Bayesian grid weights.

15. The non-transitory computer-readable medium of claim 12, wherein said extracting said multidimensional feature vector comprises assigning each component of said multidimensional feature vector a value equal to an area from zero to each successive tenth interval under a corresponding rank signal curve.

16. A system for identifying an epileptogenic zone of a subject's brain comprising a computer configured to:
receive a plurality N of electrical signals that extend over a seizure duration from a corresponding plurality N of surgically implanted electrodes in said subject's brain;
calculate within a time window components of an N×N adjacency matrix between each pair of said plurality of surgically implanted electrodes based on at least a portion of each of said plurality N of electrical signals;
calculate N eigenvectors from said N×N adjacency matrix;
select one of the N eigenvectors that has the largest eigenvalue;
assign an integer rank from 1 to N to each component of the selected eigenvector to provide an N×1 rank vector corresponding to said time window;
slide said time window by a time increment and repeat the immediately preceding three steps for the incremented time window;
repeat the immediately preceding step a plurality of times to provide said rank vector for a plurality of times, wherein each component of said rank vector corresponds to one of said N electrodes thus providing a rank signal for each of the N electrodes;
normalize each rank signal by the number of electrodes N and said seizure duration to provide corresponding normalized signals;
extract a multidimensional feature vector from each normalized signal to provide a plurality N of multidimensional feature vectors;
project each of said plurality N of multidimensional feature vectors onto a reduced dimensionality space;
receive a plurality of training data points represented in said reduced dimensionality space, each of said plurality of training data points containing information of an electrode node regarding whether brain tissue corresponding to said electrode node was resected or non-resected brain tissue and whether a corresponding surgery was successful;
calculate grid weights for each feature vector projected into said reduced dimensionality space using said training data points; and
assign a numerical value to each surgically implanted electrode using said grid weights as an indication of whether the corresponding surgically implanted electrode is in an epileptogenic zone of said subject's brain.

17. The system of claim 16, further comprising displaying a heat map based on said numerical values assigned to each surgically implanted electrode superimposed on a rendering of said subject's brain and corresponding plurality N of surgically implanted electrodes.

18. The system of claim 16, wherein said reduced dimensionality space is a smaller dimension space than a dimensionality of said multidimensional feature vectors.

19. The system of claim 18, wherein said multidimensional feature vectors are ten-dimensional feature vectors and said reduced dimensionality space is a two-dimensional space.

20. The system of any one of claims 16, wherein said calculating components of said N x N adjacency matrix between each pair of said plurality of surgically implanted electrodes uses the formula
Aᵢ(t)=[∫_{30 Hz}^{90 Hz}Pᵢ(f)Pⱼ(f)df](t)
where Pᵢ(f) and Pⱼ(f) are magnitudes of Fourier transforms of said portion of said electrical signal corresponding to said first time period from electrodes i and j, respectively, of said plurality of electrodes.

21. The system of any one of claims 16, wherein said calculating grid weights comprises calculating Bayesian grid weights.

22. The system of claim 19, wherein said extracting said multidimensional feature vector comprises assigning each component of said multidimensional feature vector a value equal to an area from zero to each successive tenth interval under a corresponding rank signal curve.
